(19) United States
(12) Patent Application Publication
(10) Pub. No.: US 2024/0156924 A1
(43) Pub. Date: May 16, 2024

(54) METHOD OF TREATING SCHIZOPHRENIA OR SCHIZOPHRENIFORM DISORDER

(71) Applicant: Curemark, LLC, Rye Brook, NY (US)

(72) Inventors: Joan M. Fallon, White Plains, NY (US); Matthew Heil, Sherman, CT (US); James Szigethy, Montgomery, NY (US); James Fallon, Armonk, NY (US)

(21) Appl. No.: 18/327,740

(22) Filed: Jun. 1, 2023

Related U.S. Application Data
(63) Continuation of application No. 17/159,846, filed on Jan. 27, 2021, now abandoned.
Application No. 17/159,846 is a continuation of application No. 16/281,908, filed on Feb. 21, 2019, now Pat. No. 10,940,187.
Application No. 16/281,908 is a continuation of application No. 15/265,415, filed on Sep. 14, 2016, now Pat. No. 10,279,016.
Application No. 15/265,415 is a continuation of application No. 14/612,604, filed on Feb. 3, 2015, now Pat. No. 9,492,515.
Application No. 14/612,604 is a continuation of application No. 14/007,793, filed on Dec. 27, 2013, now Pat. No. 8,980,252.
(60) Provisional application No. 61/477,988, filed on Apr. 21, 2011.

(51) Int. Cl. A61K 38/54; A61K 9/00; A61K 38/46; A61K 38/47; A61K 38/48; A61K 45/06; A61P 25/00

(52) U.S. Cl. CPC A61K 38/54; A61K 9/0053; A61K 38/465; A61K 38/47; A61K 38/48; A61K 45/06; A61P 25/00; C12Y 301/01; C12Y 302/01; C12Y 304/00

(57) ABSTRACT

Described herein are compositions which include digestive enzymes which are formulated to reduce one or more symptoms of a neuropsychiatric disorder. Also described herein is a method for treating an individual with a neuropsychiatric disorder using digestive enzymes and their derivatives to alleviate the symptoms of neuropsychiatric disorders. The method comprises administering to the individual an effective amount of digestive enzymes that are either naturally- or recombinantly-derived, or their derivatives, in an amount effective to reduce one or more symptoms of the neuropsychiatric disorder.

### CROSS-REFERENCE

This application is a continuation of U.S. application Ser. No. 17/159,846, filed Jan. 27, 2021, which is a continuation of U.S. application Ser. No. 16/281,908, filed Feb. 21, 2019, now U.S. Pat. No. 10,940,187, which is a continuation of U.S. application Ser. No. 15/265,415, filed Sep. 14, 2016, now U.S. Pat. No. 10,279,016, which is a continuation of U.S. application Ser. No. 14/612,604, filed Feb. 3, 2015, now U.S. Pat. No. 9,492,515, which is a continuation of U.S. application Ser. No. 14/007,793, filed Dec. 27, 2013, now U.S. Pat. No. 8,980,252, which is a § 371 U.S. National Phase Entry of PCT/US12/34489, filed Apr. 20, 2012, which claims the benefit of U.S. Provisional Application No. 61/477,988, filed Apr. 21, 2011, which application is incorporated herein by reference in its entirety.

### BACKGROUND OF THE INVENTION

Neuropsychiatric disorders are some of the most debilitating, socially isolating and economically draining of all illnesses. The manifestations of neuropsychiatric disorders, often mistaken as willful or controllable behaviors cause the illness to be misdiagnosed and thus poorly treated. Most treatments for neuropsychiatric disorders have severe and devastating side effects. These side effects will often discourage the patient from continuing treatment and are often the cause of relapse.

Psychotic disorders represent the most difficult of all neuropsychiatric disorders to control. The invariate nature of the presentation of symptoms of these disorders, together with the side effects exhibited by medications used to alleviate these symptoms, makes their treatment difficult.

The introduction of antipsychotic drugs in the 1950's heralded the “golden age” of psychopharmacology. Their development has been compared to the discovery of antibiotics for infectious diseases. Conventional or “typical” antipsychotic drugs, typified by chlorpromazine and haloperidol, have a proven track record in the treatment of schizophrenia. However, the “typical” antipsychotics described below have substantial limitations. They are most effective against the psychotic symptoms of the illness in its early stages, but their side effects are troubling and contribute significantly to non-compliance, which leads to relapse and re-hospitalization.

### SUMMARY OF THE INVENTION

There is a growing belief among clinicians that the “atypical” antipsychotics are or should become first-line treatments in schizophrenia. However, the exact nature and extent of the clinical advantages of the atypical drugs are not known. Moreover, they may cost ten times as much as older “typical” antipsychotics. Although a variety of claims of efficacy and safety have been made, they are often based on insufficient evidence.

Among the reasons for this is traditional clinical trials have excluded many patients with schizophrenia, including those who are substance abusers, violent or uncooperative, making it difficult to generalize the results of such studies to real world patients. For reasons of external validity, treatment effectiveness studies have sought to use more representative sampling techniques. However, even effectiveness studies rarely have representative samples of providers and systems of care, or large enough samples to have sufficient power to examine the role of external factors affecting treatment outcome.

There is a need for a new class of drugs to treat neuropsychiatric disorders, with at least equal effectiveness, but with a severely reduced side effect profile, which will enhance compliance and lower co-morbid symptomatology.

Provided herein are compositions of digestive enzymes which are useful in the prevention or treatment of one or more symptoms of a neuropsychiatric disorder. Also provided herein are compositions of digestive enzymes which are useful for use in the prevention or treatment of one or more symptoms of schizophrenia. Treatment of a neuropsychiatric disorder (e.g., schizophrenia) encompasses stasis of one or more symptoms (i.e., they do not worsen), as well as reduction (partial or complete) of one or more symptoms. In one embodiment, one or more symptoms of such disorders are reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%. In another embodiment, one or more symptoms of such disorders are reduced in severity or duration by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 90-fold, about 95-fold, about 100-fold, or more. Compositions may include not only one or more digestive enzymes, but also one or more pharmaceutically acceptable carriers, excipients, buffers, fillers, binders, stabilizers, surfactants, diluents, taste maskers, etc.

Provided herein is a method for using digestive enzymes and their derivatives to alleviate one or more symptoms of neuropsychiatric disorders. The method comprises administering to the individual one or more digestive enzymes that are either naturally- or recombinantly-derived, or their derivatives, in an amount effective to reduce the one or more symptoms of the neuropsychiatric disorder. Digestive enzymes generally comprise all proteases, amylases, and lipases, as well as other proteins secreted in a mammal that affect the digestive process either directly or indirectly.

The disorders that present symptoms potentially suitable for alleviation according to the present method include, but are not limited to: adjustment disorders, addiction, Alzheimer's disease, anxiety disorders, bipolar disorder, cognitive disorders, dementias, dissociative disorders, eating disorders, impulse-control disorders, mood disorders, sexual disorders, sleep disorders, psychotic disorders such as schizophrenic disorders, somatoform disorders, substance abuse disorders and personality disorders. In one embodiment, a schizophreniform disorder (e.g., schizophrenia) is treated according to the methods described herein

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with neuropsychiatric disorders.

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with schizophrenia.

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with psychosis.

In one aspect, provided herein is a method for treating an individual exhibiting one or more symptoms of a neuropsychiatric disorder, the method comprising administering a therapeutically effective amount of digestive enzymes to the individual. In one embodiment, the neuropsychiatric disorder is a schizophreniform disorder, or a mild anxiety state.

In another embodiment, a symptom of the neuropsychiatric disorder may be a positive symptom, a negative symptom, a symptom of cognitive impairment, or a combination thereof. Positive symptoms are include, but are not limited to hallucinations, delusions, disorganized thought, disorganized speech (e.g., frequent derailment or incoherence), movement disorders, bizarre behavior, and any combinations thereof. Negative symptoms include, but are not limited to limited to loss of motivation, restricted range of emotional experience and expression, reduced hedonic capacity, affective flattening, alogia, avolition, and any combinations thereof. Symptoms of cognitive impairment include, but are not limited to poor executive function, inability to use learned information, difficulty paying attention and/or focusing, and any combinations thereof.

Digestive enzymes to be used in a composition described herein include amylase, lipase, protease, and any combination thereof. In another embodiment, digestive enzymes to be used in such compositions may further include chymotrypsin, trypsin, pancreatin, papain, and any combination thereof. Digestive enzymes may be derived from a source such as, for example, animal enzymes, plant enzymes, synthetic enzymes, and any combination thereof. In a one embodiment, the animal enzyme is derived from a mammal.

Digestive enzymes may be manufactured using any appropriate technology including, but not limited to, enteric coating, lipid encapsulation, direct compression, dry granulation, wet granulation, and any combination thereof. A preparation may be an oral dosage formulation such as, for example, pills, tablets, capsules, microcapsules, mini-capsules, time released capsules, mini-tabs, sprinkles, and any combination thereof. In one embodiment digestive enzymes are provided as a pharmaceutical composition. In one embodiment the pharmaceutical composition is in the form of encapsulated sprinkles. In one embodiment the encapsulation is a lipid coating. In one embodiment the lipid coating is a soy lipid coating.

Provided herein is a method is presented for treating a symptom of a neuropsychiatric disorder in an individual comprising administering an effective amount of a composition comprising one or more digestive enzymes to the individual. In one embodiment, the neuropsychiatric disorder is schizophrenia, schizophreniform disorders, or mild anxiety states.

In one embodiment, the digestive enzyme is selected from the group consisting of amylase, lipase, protease, and any combination thereof. In another embodiment, the digestive enzyme is further selected from the group consisting of: chymotrypsin, trypsin, pancreatin, papain, and any combination thereof. In yet another embodiment, the digestive enzymes are derived from a source selected from the group consisting of animal enzymes, plant enzymes, synthetic enzymes, and any combination thereof. In one embodiment the digestive enzymes are pancreatic digestive enzymes. In one embodiment, the animal enzyme is derived from a mammal. In one embodiment the mammal is a pig. In one embodiment, digestive enzymes are derived from a mammalian pancreas. In one embodiment the pancreas is a pig pancreas.

In one embodiment, the total amount of protease in a composition ranges from about 5,000 to about 1,500,000 U.S.P. units/dose. In another embodiment, the total amount of amylase in a composition ranges from about 1,000 to about 15,000,000 U.S.P. units/dose. In another embodiment, the total amount of lipase in a composition ranges from about 1,500 to about 282,000 U.S.P. units/dose. In one embodiment a pharmaceutical composition comprises about 23,000 U.S.P. units/dose of lipase, about 144,000 U.S.P. units/dose of amylase and about 140,000 U.S.P. units/dose of protease. In another embodiment a pharmaceutical composition contains 23040 U.S.P. units/dose of lipase, about 144,000 U.S.P. units/dose of amylase and about 140,400 U.S.P. units/dose of protease.

Provided herein are methods for preventing or treating one or more symptoms of a neuropsychiatric disorder in an individual comprising, administering to said individual a composition comprising one or more digestive enzymes, wherein one or more symptoms of said neuropsychiatric disorder are partially or completely reduced. In one aspect, the neuropsychiatric disorder is a schizophreniform disorder such as, for example, schizophrenia. Symptoms of schizophrenia that may be treated with such methods include, but are not limited to, positive symptoms, negative symptoms, cognitive impairment, and any combination thereof. Positive symptoms include, but are not limited to, hallucinations, delusions, disorganized thought, disorganized speech (e.g., frequent derailment or incoherence), movement disorders, bizarre behavior, and any combination thereof. Negative symptoms include, but are not limited to, loss of motivation, restricted range of emotional experience and expression, reduced hedonic capacity, affective flattening, alogia, avolition, and any combination thereof. Symptoms of cognitive impairment include, but are not limited to, poor executive function, inability to use learned information, difficulty paying attention and/or focusing, and any combination thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1. The Test Substances.

FIG. 2. Description of the Dosages.

FIG. 3. Description of the Enzyme Components.

FIG. 4. Distance Moved by ckr Mice Treated with CM100 in Spontaneous Open Field Assays.

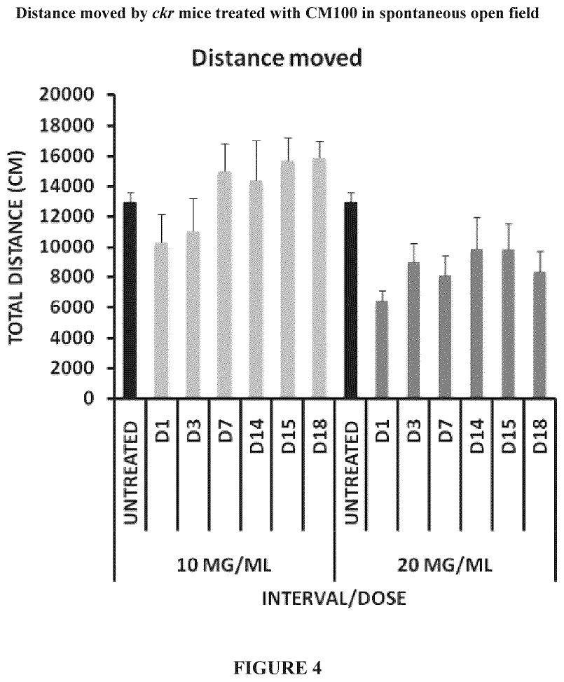

FIG. 5. Velocity of ckr Mice Treated with CM100 in Spontaneous Open Field Assays.

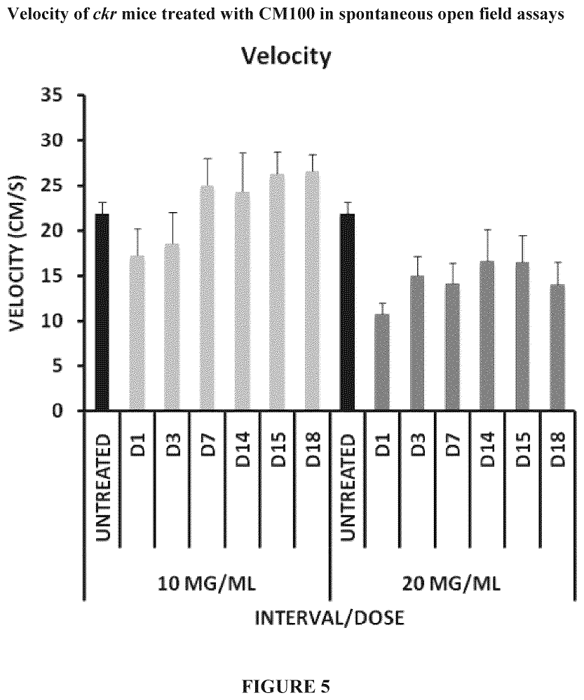

FIG. 6. Aripiprazole Dose Dependent Response in ckr Mice.

FIG. 7. Total distance moved in the 90 min recording of spontaneous open field test on male B6 mice. Data plot as Mean±SEM. Pair-wise comparison using Student's t-test. α=0.05. *, p<0.05; **, p<0.01; ***, p<0.001; N.S., not significant.

FIG. 8. Velocity in the 90 min recording of spontaneous open field test on male B6 mice. Data plot as Mean±SEM. Pair-wise comparison using Student's t-test. α=0.05. *, p<0.05; **, p<0.01; ***, p<0.001; N.S., not significant.

### DETAILED DESCRIPTION OF THE INVENTION

Provided herein are methods of treating disorders relating to neuropsychiatric disorders and methods of diagnosing the likelihood of having or developing a neuropsychiatric disorder.

Antipsychotic drugs are divided into two categories: “typical” antipsychotics and the newer “atypical” antipsychotics. Typical antipsychotics have been shunned in recent times due to their side effect profile which includes the production of extrapyramidal symptoms such a facial tics, gait disturbances, proprioceptive difficulties, rigidity, persistent muscle spasms, shakiness, restlessness, jitteriness and uncharacteristic movements.

Examples of antipsychotic drugs which fall into the “typical” category are: chlorpromazine, fluphenazine, haloperidol, loxapine, mesoridazine, molindone, perphenazine, pimozide, prochlorperazine, thiothixene, thioridazine and trifluoperazine.

With the advent of atypical antipsychotic drugs and their potential for enhanced efficacy and safety, the risk/benefit profile of this drug class has changed. Following the re-introduction of the first atypical antipsychotic, clozapine, in 1990, several new atypical drugs have become available for clinical use and now comprise more than 50% of the antipsychotic drug market in the United States. These drugs include risperidone (1994), olanzapine (1996) and quetiapine (1997).

Examples of “atypical” antipsychotic drugs are: aripiprazole, asenapine, clozapine, iloperidone, lurasidone, olanzapine, paliperidone, quetiapine, risperidone and ziprasidone among others.

Recent research has provided strong evidence of the efficacy of atypicals in schizophrenia, and demonstrated that they greatly reduce the risk of extrapyramidal side effects, as well as reducing tardive dyskinesias. “Dyskinesias” are repetitive, uncontrollable and purposeless movements of the body or face. “Tardive” refers to those symptoms that develop after long-term antipsychotic treatment (several years). Unlike early dyskinesia symptoms, tardive dyskinesias may become permanent even if the antipsychotic medication is stopped.

These “atypical” antipsychotics are not without their side effects. While they have largely replaced the older medications, atypicals are rather expensive and carry a significant risk of weight gain (sometimes extreme weight gain) and diabetes. However, they may be more effective than the “typical” antipsychotics for such conditions as schizophrenia, and other schizoaffective disorders as well as for multiple types of depression and other mental illnesses.

Although the antipsychotics were first developed for schizophrenia, antipsychotic drugs are now broadly used for other disorders, including behavioral signs and symptoms associated with Alzheimer's disease, dementias, depression, and are generally applied to the entire classes of neuropsychiatric disorders including: adjustment disorders, anxiety disorders, dissociative disorders, eating disorders, impulse-control disorders, mood disorders, sexual disorders, sleep disorders, psychotic disorders, sexual disorders, somatoform disorders, substance abuse disorders, and personality disorders. Despite their widespread use in these conditions, the overall effectiveness and safety of these drugs remain unclear.

### Neurological and Neuropsychiatric Disorders

In accordance with the present disclosure, a method is presented for alleviating symptoms of neurological disorders. In one embodiment, the method comprises administering to an individual a digestive enzyme either naturally or recombinantly derived, or their derivatives in an amount effective to reduce the symptoms of the neurological disorders.

There are over 300 neuropsychiatric disorders listed in the DSM-IV and there is overlap among the different diagnoses. Neuropsychiatric disorders are categorized according to their predominant features. For example, phobias, social anxiety and post-traumatic stress disorder all include anxiety as a main feature of the disorder. Schizophrenia and related disorders such as delusional disorder, brief psychotic disorder, schizoaffective disorder, schizophreniform disorder and shared psychotic disorder (a disorder that is characterized by the individual's inability to determine real from unreal), share similar characteristics.

In one embodiment, the neurological disorders that present symptoms potentially suitable for alleviation according to the present method include, but are not limited to the following disorders and categories of disorders: adjustment disorders, addiction, Alzheimer's disease, anxiety disorders, bipolar disorder, cognitive disorders, dementias, dissociative disorders, eating disorders, impulse-control disorders, mood disorders, sexual disorders, sleep disorders, psychotic disorders such as schizophrenic disorders (e.g., schizophrenia), somatoform disorders, substance abuse disorders and personality disorders.

As used herein, a schizophreniform disorder refers to a psychotic disorder characterized by distortions of reality and disturbances of thought and language and withdrawal from social contact.

Examples of anxiety conditions which are treated using the method of this disclosure include but are not limited to anxiety disorders, panic disorder, panic disorder with agoraphobia, panic disorder without agoraphobia, agoraphobia without history of panic disorders, social phobia, simple phobia, obsessive compulsive disorder, post-traumatic stress disorder, generalized anxiety disorder, anxiety disorder-not otherwise specified (NOS), organic anxiety disorder, psychoactive substance anxiety disorder, separation anxiety disorder, avoidant disorder of childhood or adolescence, and overanxious disorder.

Examples of neuropsychiatric conditions which are treated using the method of this disclosure include, but are not limited to, schizophrenia, catatonic, subchronic; schizophrenia, catatonic, chronic; schizophrenia, catatonic, subchronic with acute exacerbation; schizophrenia, catatonic, chronic with acute exacerbation; schizophrenia, catatonic, in remission; schizophrenia, catatonic, unspecified; schizophrenia, disorganized, subchronic; schizophrenia, disorganized, chronic; schizophrenia, disorganized, subchronic with acute exacerbation; schizophrenia, disorganized, chronic with acute exacerbation; schizophrenia, disorganized, in remission; schizophrenia, disorganized, unspecified schizophrenia, paranoid, subchronic; schizophrenia, paranoid, chronic; schizophrenia, paranoid, subchronic with acute exacerbation; schizophrenia, paranoid, chronic with acute exacerbation; schizophrenia, paranoid, in remission; schizophrenia, paranoid, unspecified; schizophrenia, undifferentiated, subchronic; schizophrenia, undifferentiated, chronic; schizophrenia, undifferentiated, subchronic with acute exacerbation; schizophrenia, undifferentiated, chronic with acute exacerbation; schizophrenia, undifferentiated, in remission; schizophrenia, undifferentiated, unspecified; schizophrenia, residual, subchronic; schizophrenia, residual, chronic; schizophrenia, residual, subchronic with acute exacerbation; schizophrenia, residual, chronic with acute exacerbation; schizophrenia, residual, in remission; schizophrenia, residual, unspecified; delusional (paranoid) disorder; brief reactive psychosis; schizophreniform disorder; schizoaffective disorder; personality disorders, schizoid; and personality disorders, schizotypal. In one embodiment, clinical features or symptoms of schizophrenia include positive symptoms, which include but are not limited to psychosis, hallucinations, delusions, disorganized thought, disorganized speech (e.g., frequent derailment or incoherence), movement disorders, and bizarre behavior. Delusions include but are not limited to false beliefs that significantly hinder a person's ability to function. For example, an individual may have the delusion that people are trying to hurt them when there is no evidence of this, or the individual having the delusion that they are somebody else. Hallucinations include false perceptions. Hallucinations may be visual (e.g., seeing things that are not there), auditory (e.g., hearing things that are not there), olfactory (e.g., smelling things that are not there), tactile (e.g., feeling sensations on the skin that are not there, such as the feeling of bugs crawling on the skin), or gustatory. Auditory hallucinations and paranoia about others reading their minds, or being able to be in their bodies are examples of symptoms experienced by schizophrenics. These symptoms appear to express an excess or distortion of normal function. Psychosis as used herein generally refers to an abnormal condition of the mind, and is a term for a mental state that may be described as a “loss of contact with reality” that is observed, in some cases, in patients with schizophrenia. Psychosis is given to the more severe forms of psychiatric disorder, during which hallucinations and delusions and impaired insight may occur.

In another embodiment, clinical features or symptoms of schizophrenia include negative symptoms, which include but are not limited to loss of motivation, restricted range of emotional experience and expression, reduced hedonic capacity, affective flattening, alogia, and avolition. Negative symptoms such as “flat affect”, lack of enjoyment, restricted communication and inability to follow through on planned activities, losing interest in everyday activities such as bathing, grooming, or getting dressed; feeling out of touch with other people, family, or friends; a lack of feeling or emotion (apathy); having little emotion or inappropriate feelings in certain situations; and having less ability to experience pleasure may be experienced. These reflect symptoms that appear to express a loss or diminution of normal function.

In yet another embodiment, clinical features or symptoms of schizophrenia include one or more symptoms of cognitive impairment. For example, cognitive impairment symptoms include poor executive function, inability to use learned information, and difficulty paying attention and/or focusing.

Individuals with neuropsychiatric disorders frequently exhibit one or more characteristics of the particular disorder. Additionally, these characteristics often overlap with symptoms of other disorders within the category of neurological disorders as well as other disorders characterized as mental illness such as, but not limited to, Alzheimer's and bipolar disorder. The present disclosure contemplates that one or more symptoms as well as a complete constellation of symptoms within one individual may be alleviated by the present method.

Recognition and determination of a reduction in symptoms of any and all of these disorders can be readily performed by those skilled in the art. One will recognize that these psychotic conditions are characterized by hallucinations, delusions or grossly disorganized behavior, which indicates that the patient suffers from gross impairment in reality.

In another embodiment, a patient treated with such methods exhibits an improvement in one or more symptoms of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% compared to a patient treated with a control substance.

A “patient” to be treated by a method described herein refers to an adult or a child. In one embodiment, a patient to be treated is a human from about 2 and about 10 years of age, from about 11 to about 20 years of age, or from about 21 to about 30 years of age.

### Diagnosis

The recognition of the symptoms of the neuropsychiatric disorder or disorders present in an individual and determination that the present method may alleviate said symptoms prior to, during, or after the practice of this method is well within the purview of an individual ordinarily skilled in the art, who can perform suitable clinical, diagnostic, and or observational or other techniques required.

The DSM IV Diagnostic Criteria for Schizophrenia is described below:
A. Characteristic symptoms: Two (or more) of the following, each present for a significant portion of time during a 1-month period (or less if successfully treated): delusions, hallucinations, disorganized speech (e.g., frequent derailment or incoherence), grossly disorganized or catatonic behavior, negative symptoms, i.e., affective flattening, alogia, or avolition. Only one Criterion A symptom is required if delusions are bizarre or hallucinations consist of a voice keeping up a running commentary on the person's behavior or thoughts, or two or more voices conversing with each other.
B. Social/occupational dysfunction: For a significant portion of the time since the onset of the disturbance, one or more major areas of functioning such as work, interpersonal relations, or self-care are markedly below the level achieved prior to the onset (or when the onset is in childhood or adolescence, failure to achieve expected level of interpersonal, academic, or occupational achievement).
C. Duration: Continuous signs of the disturbance persist for at least 6 months. This 6-month period must include at least 1 month of symptoms (or less if successfully treated) that meet Criterion A (i.e., active-phase symptoms) and may include periods of prodromal or residual symptoms. During these prodromal or residual periods, the signs of the disturbance may be manifested by only negative symptoms or two or more symptoms listed in Criterion A present in an attenuated form (e.g., odd beliefs, unusual perceptual experiences).

### Compositions and Formulations

Digestive enzymes are produced by the salivary glands, glands in the stomach, the pancreas and glands in the small intestines. Digestive enzymes produced by the pancreas are secreted into the duodenum, or upper segment of the small intestine, raising the pH to around 5 or 6, and they assist in the digestion of food components, including carbohydrates, lipids, proteins and nucleic acids.

Digestive enzymes have been administered to mammals to treat enzyme deficiencies caused by conditions affecting the pancreas, such as pancreatitis and pancreatic enzyme deficiency. Pancreatic enzymes administered to humans are commonly of porcine origin. Individuals with cystic fibrosis require the administration of enzymes, particularly lipases, in the maintenance of their condition. Manufacturers of enzyme preparations for these individuals have used enteric coatings for targeted delivery in the distal section of the small intestine, where lipase activity is important.

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with neuropsychiatric disorders.

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with schizophrenia.

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with psychosis.

In one embodiment of the present disclosure, digestive enzymes comprise proteases, amylases and lipases, as well as other proteins secreted in a mammal that affect the digestive process either directly or indirectly. In one aspect, digestive enzymes present in the composition include an amylase, a protease, or a lipase.

In another aspect, digestive enzymes present in the composition include two or more of: an amylase, a protease, and a lipase.

In one aspect, digestive enzymes present in the composition include an amylase, a protease, and a lipase.

In another aspect, a composition may further contain one or more of cellulase, papaya, bromelain, chymotrypsin, and trypsin.

In one embodiment, the digestive or pancreatic enzyme composition comprises one or more of the following: amylases, proteases, cellulase, papaya, bromelain, lipases, chymotrypsin, and trypsin.

Compositions may contain an amount of protease from about 5,000 to about 1,500,000 U.S.P. units/dose including, but not limited to about 5,000; about 7,500; about 10,000; about 15,000; about 20,000; about 25,000; about 30,000; about 40,000; about 50,000; about 65,000; about 75,000; about 100,000; about 140,000; about 140,400; about 150,000; about 200,000; about 250,000; about 300,000; about 350,000; about 400,000; about 450,000; about 465,000; about 500,000; about 550,000; about 600,000; about 650,000; about 700,000; about 750,000; about 800,000; about 850,000; about 900,000; about 950,000; about 1,000,000; about 1,050,000; about 1,100,000; about 1,150,000; about 1,200,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; and about 1,500,000 U.S.P. units/dose along with all values in between per dose.

Compositions may contain an amount of amylase from about 1,000 to about 15,000,000 U.S.P. units/dose including, but not limited to about 1,000; about 3,000; about 5,000; about 7,500; about 10,000; about 15,000; about 20,000; about 25,000; about 30,000; about 40,000; about 50,000; about 65,000; about 75,000; about 100,000; about 144,000; about 500,000; about 1,000,000; about 2,000,000; about 3,000,000; about 4,000,000; about 5,000,000; about 6,000,000; about 7,000,000; about 8,000,000; about 9,000,000; about 10,000,000; about 11,000,000; about 12,000,000; about 13,000,000; about 14,000,000; and about 15,000,000 U.S.P. units/dose, along with all values in-between per dose.

Compositions may contain an amount of lipase from about 1,500 to about 282,000 U.S.P. units/dose including, but not limited to, about 1,500; about 1,880; about 2,000; about 3,000; about 5,000; about 7,500; about 10,000; about 15,000; about 20,000; about 23,000; about 23,040; about 25,000; about 30,000; about 40,000; about 50,000; about 65,000; about 75,000; about 100,000; about 125,000; about 150,000; about 200,000; about 250,000; and about 282,000 U.S.P. units/dose along with all values in-between per dose.

In another embodiment, the digestive enzyme composition is comprised of protease, lipase, and amylase where the activities are: protease between 10,000 to 1,500,000 U.S.P. units/dose including 10,000; 100,000; 150,000; 200,000; 250,000; 300,000; 350,000; 400,000; 450,000; about 465,000; 500,000; 550,000; 600,000; 650,000; 700,000; 750,000; 800,000; 850,000; 900,000; 950,000; 1,000,000; 1,050,000; 1,100,000; 1,150,000; 1,200,000; 1,250,000; 1,300,000; 1,350,000; 1,400,000; 1,450,000; and 1,500,000 along with all values in between per dose and where the ratio of protease to lipase is such that the amount of lipase is never more than 0.188 times the amount of protease and where the ratio of protease activity to amylase activity is between 1:0.1 and 1:10.

In another embodiment a pharmaceutical composition comprises about 23,000 U.S.P. units/dose of lipase, about 144,000 U.S.P. units/dose of amylase and about 140,000 U.S.P. units/dose of protease. In another embodiment a pharmaceutical composition contains about 23,040 U.S.P. units/dose of lipase, about 144,000 U.S.P. units/dose of amylase and about 140,400 U.S.P. units/dose of protease.

In some embodiments, the digestive enzyme composition comprises at least one protease and at least one lipase, wherein the ratio of total proteases to total lipases (in U.S.P. units) ranges from about 5.371:1 to about 20:1 including 5.371:1, 6:1, 7:1, 8:1, 9:1, 10:1, 11; 1, 12; 1, 13; 1, 14:1, 15:1, 16; 1, 17:1, 18:1, 19:1 and 20:1, along with all values in-between. In some embodiments, the ratio of proteases to lipases ranges from about 5.371:1 to about 10:1 including 5.371:1, 6:1, 7:1, 8:1, 9:1, and 10:1, along with all values in-between.

In yet another embodiment, the digestive enzyme composition comprises at least one protease and at least one lipase, wherein the ratio of total proteases to total lipases (in U.S.P. units/dose) ranges from about 5.371:1 to about 20:1 including 5.371:1, 6:1, 7.1, 8.1, 9:1, 10:1, 11:1, 12:1, 13:1, 14:1, 15:1, 16:1, 17:1, 18:1, 19:1, and 20:1, along with all values in-between. In another embodiment, the digestive enzyme composition comprises at least one protease and at least one lipase, wherein the ratio of total proteases to total lipases (in U.S.P. units/dose) ranges from about 1:1 to about 20:1. In yet another embodiment, the ratio of proteases to lipases ranges from about 4:1 to about 10:1. In one embodiment, the ratio of proteases to lipases ranges from about 5.371:1 to about 10:1 including 5.371:1, 6:1, 7:1, 8:1, 9:1, and 10:1 along with all values in-between. In one embodiment, the digestive enzyme composition comprises at least one protease and at least one amylase, wherein the ratio of total proteases to total amylases (in U.S.P. units/dose) ranges from about 1:0.1 to about 1:10 including 1:0.25, 1:0.5, 1:0.75, 1:1, 1:1.25, 1:1.5, 1:1.75: 1:2, 1:1.25, 1:1.5, 1:1.75, 1:1.2, 1:1.25, 1:1.5, 1:1.75, 1:1.2, 1:1.25, 1:1.5, 1:1.75, 1:1.2: 1:1.5, 1:2, 1:3, 1:4, 1:5, 1:6, 1:7, 1:8, 1.9 and 1:10 along with all values in-between.

The disclosure also relates to a specific blend of enzymes, with or without coating, with or without other components as described above whereby enzyme administration occurs in individuals with a neurological or neuropsychiatric disorder, including but not limited to: adjustment disorders, addiction, Alzheimer's disease, anxiety disorders, bipolar disorder, cognitive disorders, dementias, dissociative disorders, eating disorders, impulse-control disorders, mood disorders, sexual disorders, sleep disorders, psychotic disorders such as schizophrenic disorders (e.g., schizophrenia), somatoform disorders, substance abuse disorders and personality disorders. Recognition and determination of a reduction of symptoms of any and all of these disorders can be readily performed by those skilled in the art using conventional assays.

In one embodiment of the present disclosure, uncoated digestive enzymes comprise proteases, amylases and lipases, as well as other proteins secreted in a mammal, which affect the digestive process either directly or indirectly. In one embodiment, the digestive or pancreatic enzyme composition comprises one or more of the following: amylases, proteases, cellulase, papaya, bromelain, lipases, chymotrypsin, and trypsin.

In one embodiment the coated or uncoated digestive enzymes to be administered are comprised of pancreatin, pancrelipase, or a combination thereof. In one embodiment a coating technology can be used, such as the ones described in U.S. Pat. Nos. 6,835,397, 6,153,236, or US 2009-0004285 which are herein incorporated by reference in their entirety.

Enzyme preparations with non-lipid enteric coatings can be used to deliver lipases in individuals in need of lipase administration. Certain methods and enzyme compositions for use in treating children and other individuals in, for example, U.S. Pat. Nos. 7,138,123, 6,660,831, 6,632,429, 6,534,063, which is herein incorporated by reference in its entirety.

The composition of the dosage form may include other components, generally utilized in pharmaceutical preparations including but not limited to binders, disintegrants, extracts, lubricants, fillers, flavorings, preservatives, colorants, taste maskers, diluents and coating agents, such as vegetable oil, crystalline oils, and other coating methodologies.

In one embodiment, coating of a digestive enzyme preparation is used to obtain release at selected transit times or in selected locations of the gastrointestinal tract of humans. In one aspect, this disclosure relates to controlled release enzyme preparations administered to an individual with a neuropsychiatric disorder.

In yet another aspect, this disclosure relates to an enzyme delivery system comprising a coated enzyme preparation having particles which comprise: (a) a core comprising pancreatic or digestive enzymes present in an amount from about 5% to 99% by weight of the particles; and (b) a generally uniform coating to provide for controlled release of the enzymes, said coating comprising an emulsifiable lipid. In one aspect, the coated enzyme preparation particles of the enzyme delivery system are non-aerosolizable.

In some embodiments a coated digestive enzyme preparation comprising (a) a core containing a digestive enzyme particle, where the enzyme present in an amount of from about 5% to 95% by weight of the particles, including 5%, 10%, 15%, 20%, 25%, 30%, 35%, 40%, 45%, 50%, 55%, 60%, 65%, 70%, 75%, 80%, 85%, 90% and 95% by weight, along with all values in-between; and (b) a coating comprising a crystallizable lipid, wherein the coating continuously coats the core and the crystallizable lipid releases the enzyme upon exposure to physiological conditions.

In some embodiments a coated enzyme preparation having particles which comprise: (a) a core comprising pancreatic or other digestive enzymes present in an amount of from about 5% to 95% by weight of the particles, including 5%, 10%, 15%, 20%, 25%, 30%, 35%, 40%, 45%, 50%, 55%, 60%, 65%, 70%, 75%, 80%, 85%, 90% and 95% by weight along with all values in-between; and (b) a generally uniform coating to provide for controlled release of the enzymes, the coating comprising a crystallizable lipid. In some embodiments, the coated enzyme preparation particles of the enzyme delivery system are non-aerosolizable.

The present disclosure also relates to methods of making the enzyme preparations by lipid coating and/or encapsulation of digestive enzymes. The methods comprise providing an emulsifiable lipid, and coating pancreatic/digestive enzyme particles with the lipid. The digestive enzymes comprise 5 to 99% of the coated enzyme preparations by weight.

In another aspect as described herein, the inventors have discovered that the methods of this disclosure produce coated digestive enzyme preparations comprising digestive and/or pancreatic enzymes coated with an emulsifiable lipid alone, or with a lipid blend to achieve a controlled rate of enzyme release, with increased release of the pancreatic/digestive enzyme upon exposure of the coated preparation to a suitable solvent. The inventors have discovered that coated pancreatic/digestive enzyme preparations having a coating consisting essentially of one or more monoglycerides exhibit time-sensitive biologically-suitable release of the pancreatic/digestive enzymes upon exposure of the coated composite to a solvent, such as water, while protecting against release in 0.1 N HCl or acidic gastric fluid.

The nature of the human digestive tract creates challenges for the delivery of digestive enzymes to patients susceptible to treatment with digestive enzymes. Multiple temperature and pH changes over the course of the digestive tract make specific delivery a challenge but a necessity. For instance, pH as low as 1 is encountered in the stomach, but rapidly increases to a more basic pH of 5-6 in the proximal small intestine. For example, generally the pH in the stomach is approximately 1.2, the pH in the duodenum is about 5.0 to 6.0; the pH in the jejunum is about 6.8, and the pH is about 7.2 in the proximal ileum and about 7.5 in the distal ileum. The low pH in the stomach that changes rapidly to a more basic pH of 5-6 in the proximal small intestines calls for a specific delivery method depending upon where the enzyme is to be delivered.

Delivery of digestive enzymes can also be challenging due to the rapid degradation and denaturing of enzymes at ambient room temperature, as well as the enhanced degradation and denaturing that can occur with high temperature, pressure, humidity and/or exposure to light. Moisture and heat together can quickly destabilize enzymes, reducing their effectiveness, and weaken their potency leading to inaccurate dosing and shortened shelf life. Denaturation or destabilization of the enzymes can reduce their effectiveness by reducing the dose of active enzymes to less than the amount needed for effective treatment. In one embodiment, to protect and stabilize the pancreatic/digestive enzyme from unfavorable conditions such as oxidation, the pancreatic/digestive enzyme (core) is coated or encapsulated in a continuous coating containing an emulsifiable lipid. In another aspect, this disclosure provides new coated enzyme preparations with improved shelf life.

Manufacturers of enzyme preparations have used enteric coatings to deliver lipases in individuals requiring administration of lipases, such as individuals with cystic fibrosis. Because the porcine enzymes are delivered in a mixture of proteases, lipases and amylases, and because these compositions for human consumption were prepared for lipase delivery, the uses of these enteric coatings, which include such substances as hypromellose phthalate, dimethicone 1000, and dibutyl phthalate, preclude delivery of proteases at the proper location for protein digestion, which is the duodenum. All other enzyme preparations presently on the market contain at least one of these enteric coating substances and/or other additives in the preparation.

In one embodiment the present disclosure includes a coated digestive enzyme preparation and/or composite, which in some embodiments is an encapsulated pancreatic/digestive enzyme preparation. In other aspects, the disclosure includes enzyme delivery systems and pharmaceutical compositions comprising coated pancreatic/digestive enzyme preparations. These coated or encapsulated enzyme preparations contain cores comprising pancreatic or digestive enzyme particles, and a coating comprising an emulsifiable lipid.

The coatings in the digestive/pancreatic enzyme preparations create a barrier to degradation and denaturation, and allow more accurate levels of active enzymes to be utilized by treated individuals. The lipid coating of this disclosure provides a significant barrier to moisture, humidity and exposure to light by allowing for a physical barrier as well as one that prevents and/or reduces hydrolysis. The coated enzyme preparations undergo less hydrolysis as a result of protection from moisture in the environment by the lipid coating. As a result of the present disclosure, pancreatic/digestive enzymes are provided which can tolerate storage conditions (e.g., moisture, heat, oxygen, etc.) for long periods of time thus enabling extended shelf life. The coating of the encapsulated enzyme preparation protects the enzyme from the environment and provides emulsification in a solvent without detracting from the abrasion resistance of the coating. The disclosure thus further relates to more stable enzyme preparations.

It is another aspect of the present disclosure to make an enzyme preparation without the use of extenders colorants, dyes, flow enhancers and other additives to reduce the potential for allergens and other sensitivity reactions in children and other treated individuals. It has been discovered that in some embodiments, the digestive enzymes can be encapsulated with a single lipid excipient to improve retention of enzyme activity, ease of administration, tolerability, and safety of administration, among other properties. Surprisingly, digestive enzyme particles containing lipases can be successfully encapsulated with coating consisting essentially of only hydrogenated soy oil.

Porcine pancreatic/digestive enzymes possess a significant odor and taste, similar to cured or smoked pork. This taste and smell can be strong and offensive to some individuals taking enzyme replacement, and especially to children. In one embodiment, the addition of a lipid coating provides significant odor and taste masking to the enzyme preparation, which allows for the tolerance of taste, as the lipid coating is odorless and tasteless. The use of this method of taste masking not involving the use of color, dyes, perfumes or other substances is preferable for the administration of medications, which have an unpleasant or undesirable taste and odor. In another embodiment, this disclosure relates to coated digestive enzyme preparations with improved taste and odor.

In some embodiments, the coatings on the digestive enzyme particle cores are preferably continuous coatings. By “continuous”, it is meant that the pancreatic/digestive enzyme is completely surrounded. The continuous coating fully surrounds or encapsulates the pancreatic/digestive enzymes. The encapsulation provides protection of the pancreatic/digestive enzyme from conditions such as moisture and oxidation.

In the manufacture of pharmaceuticals, encapsulation refers to a range of techniques used to enclose medicines in a relatively stable shell known as a capsule, allowing them to, for example, be taken orally or be used as suppositories. “Encapsulate” as used herein means that the coating completely surrounds the pancreatic/digestive enzyme. A coated or encapsulated preparation may contain one or more digestive enzyme particles enveloped in one coating to form one coated or encapsulated digestive enzyme particle in the coated or encapsulated digestive enzyme preparation.

The two main types of capsules are hard-shelled capsules, which are normally used for dry, powdered ingredients, and soft-shelled capsules, primarily used for oils and for active ingredients that are dissolved or suspended in oil. Both of these classes of capsule are made both from gelatin and from plant-based gelling substances like carrageenans and modified forms of starch and cellulose, and the latter form is usually seamless. Capsules are made in two parts by dipping metal rods in molten gelatin solution. The capsules are supplied as closed units to the pharmaceutical manufacturer. Before use, the two halves are separated, the capsule is filled with powder (either by placing a compressed slug of powder into one half of the capsule, or by filling one half of the capsule with loose powder) and the other half of the capsule is pressed on. The advantage of inserting a slug of compressed powder is that control of weight variation is better, but the machinery involved is more complex.

Sprinkle capsules are a dosage form consisting of small beads or granules of an active drug contained in a capsule that can be readily administered by simply opening up the capsule and distributing the contents over something to be swallowed.

In addition, the encapsulation also provides controlled release of the pancreatic/digestive enzyme. In one embodiment, the emulsification properties of the coating in a solvent allows for controlled release of the enzyme in the gastrointestinal (GI) system, preferably the region of the GI tract where the enzymes are to be utilized. For example, for conditions requiring treatment with proteases, the release of the protease portion of the enzymes is necessary in the proximal small intestine, thereby necessitating a lipid encapsulation, which has a dissolution profile showing a release of between 10% to 100% of the active substance into solution over a time period of between 30 and 90 minutes. In one embodiment, the dissolution profile shows a release of about 20%, 30%, 40%, 50%, 60%, 70%, 80%, 90% or 100%, and all values in between, of the coated substance into solution over a time period of about 30, 35, 40, 45, 50, 55, 60, 65, 70, 75, 80, 85 or 90 minutes and all values in between. Dissolution profiles may be obtained using methods and conditions known to those of skill in the art. For example, dissolution profiles can be determined at various pHs, including pH 1, 2, 3, 4, 5, 6, 7, 8, 9, 10 and all values in between.

The rate of release of the bioactive substance can also be controlled by the addition of additives as described below. When the preparations are exposed to a solvent, the solvent interacts with the mollifiable lipid in the coating and results in emulsification of the coating and release of the bioactive substance.

A suspension is a heterogeneous fluid containing solid particles that are sufficiently large for sedimentation. Usually they must be larger than 1 micrometer. The internal phase (solid) is dispersed throughout the external phase (fluid) through mechanical agitation, with the use of certain excipients or suspending agents. Unlike colloids, suspensions will eventually settle. An example of a suspension would be sand in water. The suspended particles are visible under a microscope and will settle over time if left undisturbed. This distinguishes a suspension from a colloid in which the suspended particles are smaller and do not settle. Colloids and suspensions are different from a solution, in which the dissolved substance (solute) does not exist as a solid and solvent and solute are homogeneously mixed. Oftentimes, powders of active ingredients may be packaged such that the addition of a diluent dissolves the powder and holds it in a liquid suspension.

A pharmaceutical preparation may be prepared in which an excipient provides a matrix to capture and protect a product before delivery. Pharmaceutical preparations may be prepared whereby the individual who takes the preparation has a reduction in the number of capsules/tablets per dosage; i.e., the preparation is stabilized and may contain a therapeutically effective amount of a protease, an amylase, and/or a lipase. Preparations may include, for example, a stabilizing matrix consisting essentially of a solidified microcrystalline cellulose which captures and protects therapeutically effective amounts of digestive enzyme particles within the stabilizing matrix. This can be done, for example, through the use of what is known in the art as PROSOLV® technology.

PROSOLV® is a combination of excipients which allow for optimized flow, compaction and product uniformity. This technology allows for uniformity in this combination, as well as manufacturing a very small tablet which would be amenable for children. With PROSOLV® technology, the ingredients are not just blended, but are co-processed, which assures that equal particles are uniformly distributed and these results are easily reproducible. This allows for stability and superb product quality.

Whether utilizing the PROSOLV® method or other methodology, the one or more digestive enzymes will be formulated and manufactured such that the particles will be uniformly distributed and there will be no overage with respect to the amount of enzyme found in the preparation. Said new drug formulation can be found in, but is not limited to, formulations which include digestive/pancreatic enzymes with and without the utilization of the PROSOLV® technology.

In a further embodiment, a direct compression method may be used for the manufacture of a pharmaceutical tablet preparation including the steps of: (a) forming an active blend by blending an intimate admixture of silicified microcrystalline cellulose and a therapeutic agent comprising one or more digestive enzymes; (b) forming a color blend by blending an intimate admixture of one or more pharmaceutically acceptable dyes and silicified microcrystalline cellulose if color is necessary; (c) combining the active blend, the color blend and a disintegrant into a pre-blend; (d) adding a lubricant to the pre-blend to form a final blend; and (e) compressing the final blend to form a pharmaceutical tablet preparation or a mixture of time released microtabs or a time released tablet.

This may be accomplished by combining the digestive enzymes with one of the patented PROSOLV® technologies, i.e.: PROSOLV® SMCC 50 or PROSOLV® SMCC 90, or other PROSOLV® technologies. When employing the PROSOLV® method, the silicified microcrystalline cellulose (SMCC) used in the preparation of the present invention may be any commercially available combination of microcrystalline cellulose granulated with colloidal silicon dioxide. The SMCC generally will be as described in Sherwood et al, Pharm. Tech., October 1998, 78-88 and U.S. Pat. No. 5,585,115, which is incorporated herein by reference in its entirety. SMCC can be obtained commercially from Edward Mendell Company, Inc., a subsidiary of Penwest Ltd., under the name PROSOLV® SMCC. There are different grades of SMCC available, with particle size being the differentiating property among the grades. For example, PROSOLV® SMCC 90 has a median particle size, by sieve analysis, in the region of 90 micrometers. PROSOLV® SMCC 50 has a median particle size, by sieve analysis, in the region of about 40-50 micrometers.

A pharmaceutical composition described herein may be prepared using a direct compression method, a dry granulation method, or by wet granulation. Preferably, the digestive/pancreatic enzyme preparation may be prepared using a direct compression process. This preferred process consists of two main steps: blending and compression.

The blending step is composed of an active blend, color blend, pre-blend, and final blend (lubrication). The formulation of the present invention may include a number of other ingredients for optimal characteristics of the pharmaceutical composition. Such other ingredients and the amounts to be used are within the knowledge of one in the art and are known in the pharmaceutical arts. These may include disintegrates, lubricants and/or coloring agents among others. Suitable disintegrants include, for example, sodium starch glycolate, other starches such as pregelatinized starch, and celluloses. Suitable lubricants may be provided, such as magnesium stearate, calcium stearate, talc and stearic acid. Any coloring agent certified by the FDA may be used, such as FD&C Yellow #6, among others.

When used as a pharmaceutical preparation, elixirs contain an active ingredient that is dissolved in a solution that contains some percentage (usually 40-60%) of ethyl alcohol and is designed to be taken orally.

Syrups are oftentimes employed as a base for medicinal purposes and consist of a concentrated or saturated solution of refined sugar in distilled water.

A suspension of liquid droplets or fine solid particles in a gas is called an aerosol. This can take the form of an oral spray.

A gum may be devised whereby an active ingredient is incorporated into a vegetative resinous substance (e.g., acacia) and released via the actual mechanical effect of chewing or the action of saliva on the gum itself.

A thinstrip is an active pharmaceutical product coated by a lipid layer designed to dissolve in the mouth over a brief period of time. The same technology could be used to produce a medicated lollipop for transmucosal delivery.

In pharmaceutical terms, a granule is a small particle gathered into a larger, permanent aggregate in which the original particles can still be identified.

In some aspects, the disclosure relates to the production of selected coated enzyme preparations made by coating digestive enzyme particles with lipids not previously used in coated digestive enzyme preparations. The unique mixtures of emulsifiable lipids and enzymes can deliver certain components of the pancreatic/digestive enzymes to selected locations and/or at selected times during transit of the GI tract. In some aspects, the disclosure relates to methods of delivering digestive enzymes to humans based upon dissolution profiles.

The emulsifiable lipid may be any lipid, lipid mixture, or blend of lipid and emulsifiers which emulsifies when exposed to a solvent, and has a melting point which allows the lipid to be a solid at typical storage temperatures. The emulsifiable lipid can be a vegetable or animal derived-lipid. In another embodiment, the emulsifiable lipid consists essentially of, or comprises one or more monoglycerides, diglycerides or triglycerides, or other components including, for example, emulsifiers found in hydrogenated vegetable oils. In another embodiment the lipid is a non-polar lipid.

As used herein, animal and/or vegetable “derived” lipids can include fats and oils originating from plant or animal sources and/or tissues, and/or synthetically produced based on the structures of fats and oils originating from plant or animal sources. Lipid material may be refined, extracted or purified by known chemical or mechanical processes. The lipid may, in one embodiment, comprise a Type I USP-National Formulary vegetable oil.

The digestive enzyme used in the present disclosure can be any combination of digestive enzymes of a type produced by the pancreas, including, but not limited to digestive enzymes from a pancreatic source or other sources. The scope of the disclosure is not limited to pancreatic enzymes of porcine origin, but can be of other animal or plant origin as well as those that are synthetically derived. In one embodiment, the digestive enzyme is derived from mammalian sources such as porcine-derived digestive enzymes. In another embodiment, the enzyme includes one or more enzymes, and is plant derived, synthetically derived, recombinantly produced in microbial, yeast, or mammalian cells, or includes a mixture of enzymes from one or more sources. For example, digestive enzymes may include one or more enzymes from one or more sources mixed together. This includes, for example, the addition of single digestive enzymes to digestive enzymes derived from pancreatic sources in order to provide appropriate levels of specific enzymes that provide more effective treatment for a selected disease or condition. One source of digestive enzymes can be obtained, for example, from Scientific Protein Laboratories. In one embodiment, the digestive enzyme is, for example a pancreatin/pancrelipase composition. In another embodiment, the digestive enzymes comprise or consist essentially of 25 U.S.P. units protease, 2 U.S.P. units lipase, and 25 U.S.P. units amylase per milligram. The term digestive enzyme may refer to one or more enzymes of a type produced by the pancreas.

In one embodiment, the digestive enzyme used present as consisting of particles having various sizes. In another embodiment, the particles of digestive enzyme are screened to obtain particles of a suitable size for encapsulation by removing particles that are too fine or too large. For example, the particles may be sieved to obtain particles of a suitable size or more uniform size range for encapsulation.

In one embodiment, the minimum amount of pancreatic enzyme present in the core is at least about 5% active enzymes by weight of the coated enzyme preparation, but in another embodiment is at least about 30%, or at least about 50% by weight. In one embodiment, the maximum amount of pancreatic/digestive enzyme present in the composite is at most about 99% by weight, and in another embodiment is at most about 98%, 95%, 90%, 85%, 80%, 75% or 70% of the coated enzyme preparation. In another embodiment, the amount of pancreatic enzyme present in the composite is about 10%, 15%, 20%, 25%, 35%, 40%, 45%, 55%, 60%, 65%, 70%, 72.5%, 75%, 77.5%, 80%, 82.5%, 87.5%, or 92.5% by weight or anywhere in between. At least about or at most about a % of enzyme may include equal to or about that % of enzyme. The term “about” includes equal to, and a range that takes into account experimental error in a given measurement. As used in connection with particle sizes, the term “about” can refer to plus or minus 10, 9, 8, 7, 6, 5, 4, 3, 2 or 1% or anywhere in between. As used in connection with % particles that can be sieved, the term “about” can refer to plus or minus 10, 9, 8, 7, 6, 5, 4, 3, 2 or 1% or anywhere in between.

In one embodiment, the composition which contains the encapsulated digestive enzyme preparation or composite is delivered as a sprinkle, powder, capsule, tablet, pellet, caplet or other oral form. In another embodiment, packaging the encapsulated enzyme preparations in an enzyme delivery system that further comprises single dose sachet-housed sprinkle preparations allows for ease of delivery and accurate dosing of the enzyme by allowing a specific amount of enzyme to be delivered in each dosing. Allowing for specific unit dosing of an enzyme preparation which maintains the enzyme activity within specific stability parameters is an enhancement over other sprinkle formulations, which are housed in a multi-unit dosing form that allows for air, moisture and heat to depredate and denature the enzyme preparation. In one embodiment, the powder or sachet is housed in a trilaminar pouch of which one layer is foil, or similar barrier to keep out moisture and to protect the enzyme preparation from adverse environmental factors. The disclosure further relates to an improvement in stability due to a reduction in hydrolysis due to the lipid encapsulation and composition of package.

In another embodiment, the lipid encapsulation methodology reduces the aerosolization of the enzyme preparation that may be caustic to the patient if inhaled. In another embodiment, the disclosure includes delivery of digestive enzymes with improved safety of administration, by reducing the amount of aerosolization of the enzyme. The lipid encapsulation reduces aerosolization and the potential for caustic burn, aspiration, and/or aspiration pneumonias in patients and administrators of the enzyme preparation, thereby reducing the potential for illness in already compromised children such as those with cystic fibrosis, and leading to safer administration.

As used herein, the term “non-aerosolizable” will be used to refer to a coated or encapsulated enzyme preparation where substantially all of the particles are large enough to eliminate or reduce aerosolization upon pouring of the coated enzyme preparation compared to uncoated enzyme particles.

As described and referred to herein, suitable pancreatic/digestive enzymes and suitable coatings may be used in the compositions and methods of this disclosure. The choice of suitable enzymes and of suitable lipid coatings, including choice of the type or amount of enzymes or coating, are guided by the specific enzyme needs of the individuals, and the selected diseases to be treated. The encapsulated enzyme preparations that are one aspect of this disclosure have not been previously described.

In another embodiment, the disclosure relates to a method of controlling the rate of release of the pancreatic/digestive enzyme from an encapsulated enzyme preparation upon exposure to a solvent. In one aspect, the method comprises blending an emulsifiable lipid with an amount of one or more additives to obtain a lipid blend and coating the digestive enzyme particle with the blend to form an encapsulated digestive enzyme preparation containing particles comprising a core which contains the enzyme, and a coating which contains the lipid. In one embodiment, the emulsifiable lipid is a blend where the emulsifiable lipid and additive are not the same, and where the rate of release of the enzyme from the encapsulated composite upon exposure to a solvent is decreased as the amount of additive is increased. In the alternative, the rate of release of the enzyme from the encapsulated composite upon exposure to a solvent is increased as the amount of additive is decreased.

The lipid coating surprisingly does not appear to be reduced or destroyed by hydrochloric acid (HCl) present in the stomach, thereby protecting the enzyme from degradation following administration until the enzyme preparation reaches its target region in the GI tract. Further the lipid coat reduces the exposure of the enzyme to attack by water, thereby reducing hydrolysis, and further protecting the digestive enzymes from degradation. In addition, the inventors have found that an excipient containing only lipid can be used to coat or encapsulate digestive enzyme particles containing lipase.

Enzyme preparations supplied by the API supplier may be provided as irregular shaped, and multi-sized particles, with uneven edges, and much clumping, and containing some crystalline salt particles. Uneven particle size and shape reduces flow properties, and interferes with packaging. In addition, pouring uncoated enzyme into the mouth of an individual would be difficult, and potentially may cause too much or too little of the enzyme to be delivered. In one embodiment, processing the digestive enzyme particles according to methods in accordance with one aspect of this disclosure yields a non-dusty, free-flowing particulate preparation suitable for sachet packaging and for pouring onto food or drink. In addition, as discussed throughout, the use of lipid encapsulation to prevent aerosolization and, therefore, increase safety, and to increase flow properties which enhance manufacturing of a pharmaceutical is an embodiment of the instant disclosure.

“Emulsifiable lipids” as used herein means those lipids that contain at least one hydrophilic group and at least one hydrophobic group, and have a structure capable of forming a hydrophilic and hydrophobic interface. These chemical and/or physical properties, mentioned above of an emulsifiable lipid permit emulsification. Examples of interfaces include, for example, micelles and bilayers. The hydrophilic group can be a polar group and can be charged or uncharged.

In one embodiment, the emulsifiable lipid is derived from animal or vegetable origins, such as, for example, palm kernel oil, soybean oil, cottonseed oil, canola oil, and poultry fat, including hydrogenated type I vegetable oils. In one embodiment, the lipid is hydrogenated. In another embodiment, the lipid is saturated or partially saturated. Examples of emulsifiable lipids include, but are not limited to, monoglycerides, diglycerides, fatty acids, esters of fatty acids, phospholipids, salts thereof, and combinations thereof.

The emulsifiable lipid is preferably a food grade emulsifiable lipid. Some examples of food grade emulsifiable lipids include sorbitan monostearates, sorbitan tristearates and calcium stearoyl lactylates. Examples of food grade fatty acid esters which are emulsifiable lipids include acetic acid esters of mono- and diglycerides, citric acid esters of mono- and di-glycerides, lactic acid esters of mono- and digylcerides, polyglycerol esters of fatty acids, propylene glycol esters of fatty acids, and diacetyl tartaric acid esters of mono- and diglycerides. Lipids can include, for example, hydrogenated soy oil. Any emulsifiable lipid may be used in the methods and products of this disclosure. In one embodiment, the emulsifiable lipid used will produce non-agglomerating, non-aerosolizing enzyme preparation particles.

In another embodiment, the method relates to preparation of an encapsulated, controlled release digestive enzyme preparation with enhanced flow properties useful in the treatment of individuals with a neurological or neuropsychiatric disorder, the method comprising: a) blending an emulsifiable lipid with one or more additives to obtain a blend; and b) coating screened digestive enzyme with the blend to form an encapsulated digestive enzyme containing a core which contains the digestive enzyme and a coating which contains the blend of emulsifiable lipid.

The coating of the enzyme with the lipid allows for the enzyme to become more uniform in size and shape, but reduces the jagged edges associated with the raw enzyme, and allows for ease of administration and ease of packaging, as the flow properties associated with the covered enzyme will allow for the packaging machinery to easily fill the sachet/pouch with the enzyme and reduces overfilling or underfilling of the sachet.

In another embodiment, the disclosure relates to a method of controlling the rate of release of a digestive enzyme from the encapsulated preparation by using a lipid blend to coat the digestive enzyme. The method includes blending an emulsifiable lipid with one or more additives to obtain a blend, and coating the digestive enzyme with the blend to form an encapsulated digestive enzyme containing a core which contains the digestive enzyme and a coating which contains the blend of emulsifiable lipid. The rate of release of the enzyme from the encapsulated preparation upon exposure with a solvent is decreased as the amount of additive is increased. In the alternative, the rate of release of the enzyme from the encapsulated composite upon exposure with a solvent is increased as the amount of additive is decreased. Thus, the nature of the coating allows for controlled release of the enzyme from the encapsulate.

Different dosage forms have different benefits. Tablets and capsules are the most common dosage forms for oral administration due to ease of manufacture, packaging and administration. Different forms of tablets have been primarily devised to meet the needs of select populations while maintaining the integrity of the active pharmaceutical ingredient. Some populations, notably infants and young children, cannot swallow tablets or capsules or find it difficult to do so. In these instances, a tablet that dissolves under the tongue, in the mouth, or in a specified liquid, or one that could be harmlessly chewed would be beneficial. Capsules that could be opened and their contents sprinkled over a small amount of food or in a liquid would also be beneficial. Any improvement that eases the administration of a necessary medication or lessens the antagonism associated with said administration, without compromising the effectiveness of the active pharmaceutical ingredient, is worthwhile.

Other types of solid dosage forms such as thin strips, lollipops or gum bring a novel concept to the administration of medications to children. Aside from the obvious ease of administration from the viewpoint of the caregiver, there may be an added benefit. The administration of medication is oftentimes a private issue and the ability of a caregiver to provide a dose of medication in a seemingly matter-of-fact form may preserve that perception and instill in the user a mindset that views the administration as pleasant rather than monotonous or negative.

Liquid dosage forms also provide benefits of administration to infants and young children or anyone with compromised swallowing capability. Syrups, solutions and suspensions are easily swallowed. Unpleasant tastes can be masked by flavoring. An oral spray allows for the quick administration of a pre-measured dose of medication and supplies multiple metered doses in one handy device. With no need to aid swallowing (such as a glass of water, etc.) and the convenience of not having to rifle through a bottle of tablets, administration is simplified.

A tablet is a mixture of active substances and excipients, usually in powder form, pressed or compacted into a solid. The excipients include binders, glidants (flow aids) and lubricants to ensure efficient tableting; disintegrants to ensure that the tablet breaks up in the digestive tract; sweeteners or flavors to mask the taste of bad-tasting active ingredients; and pigments to make uncoated tablets visually attractive. A coating (sugar, enteric or film) may be applied to hide the taste of the tablet's components, to make the tablet smoother and easier to swallow, and to make it more resistant to the environment, extending its shelf life. Tablets may be buffered (by potassium metaphosphate, potassium phosphate, monobasic sodium acetate, etc.) to combat change in pH. Tablets may be delayed-release, sustained-release, extended-release, controlled-delivery, long-acting, orally-disintegrating or melts, among others, often denoting the pharmacokinetic profile of the active agent. A capsule-shaped tablet is a caplet.

Some tablets may be taken sublingually or allowed to dissolve in the mouth. The principle behind sublingual administration is simple. When a chemical comes in contact with the mucous membrane beneath the tongue, or buccal mucosa, it diffuses through it. Because the connective tissue beneath the epithelium contains a profusion of capillaries, the substance then diffuses into them and enters the venous circulation. Troches are medicated lozenges designed to dissolve in the mouth. Soluble tablets dissolve on contact with the tongue.

Slurry may be made when a dissolvable tablet containing a gelling agent is added to a liquid.

Tablets may also be micro-coated and placed in a capsule for administration.

The compositions described herein can be administered either alone or more typically in combination with one or more of a conventional pharmaceutical carrier, excipient buffer, stabilizer or the like. Such materials should be non-toxic and should not interfere with the efficacy of the active ingredient. The precise nature of the carrier or other material will depend on the route of administration. The term “excipient” is used herein to describe any ingredient other than the compound(s) (enzymes) used in the composition as described herein and known in the art.

Acceptable carriers are physiologically acceptable to the administered patient and retain the therapeutic properties of the compounds with/in which it is administered. Acceptable carriers and their formulations are and generally described in, for example, Remington' pharmaceutical Sciences (18th Edition, ed. A. Gennaro, Mack Publishing Co., Easton, Pa. 1990). Two exemplary carriers are water and physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site to a portion of the body. Each carrier is acceptable in the sense of being compatible with the other ingredients of the formulation and not injurious to a subject to whom it is administered. Nor should an acceptable carrier alter the specific activity of the subject compounds.

Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as TWEEN®, PLURONICS® or polyethylene glycol (PEG).

The phrase “pharmaceutically acceptable” refers to molecular entities and compositions that are physiologically tolerable and do not typically produce an allergic or similar untoward reaction, such as gastric upset, dizziness and the like, when administered to a human.

### Dosing, Administration and Methods

Methods of preparing such dosage forms are known, or will be apparent, to those skilled in this art; for example, see Remington: The Science and Practice of Pharmacy, 21st Edition (Lippincott Williams & Wilkins. 2005). Appropriate dosages will depend on the patient (age, weight, overall health, etc., the severity of the condition, the type of formulation and other factors known to those having ordinary skill in the art. It is to be noted that concentrations and dosage values can vary with the severity of the condition. It is to be further understood that for any particular patient, specific dosage regimens should be adjusted over time according to the individual need and the professional judgment of the person administering or supervising the administration of the compositions.

In one embodiment a composition can be administered 1 or more times a day, such as 1, 2, 3, 4, 5, 6, 7, 8, 9, or 10 times a day with or without food. In another embodiment, a composition can be administered orally 3 times a day with or without food.

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

Provided herein are methods for administering the enzyme compositions/preparations. In one aspect, the methods include administering the pancreatic/digestive enzymes as coated preparations. In another aspect, the disclosure relates to a method of treatment comprising administering to a subject with a neurological or neuropsychiatric disorder, including but not limited to: adjustment disorders, addiction, Alzheimer's disease, anxiety disorders, bipolar disorder, cognitive disorders, dementias, dissociative disorders, eating disorders, impulse-control disorders, mood disorders, sexual disorders, sleep disorders, psychotic disorders such as schizophrenic disorders (e.g., schizophrenia), somatoform disorders, substance abuse disorders and personality disorders, or other behavioral or neurological condition in need of treatment with digestive enzymes, at least two doses of a composition comprising a therapeutically effective amount of a coated or uncoated digestive enzyme preparation comprising a core comprising a digestive enzyme. Determination of whether a subject is in need of treatment with an effective amount of digestive enzymes may be based on a determination that the subject has an enzyme deficiency.

In one aspect of the present disclosure, it is well known that determining a dosage regimen of the compound is well within the purview of those in the art. By way of example, the dose levels may range from 100 milligrams to 10 grams as determined by weight. Further activity of the enzymes may range from 100 units of activity to 1,500,000 units of activity per dose for amylases, lipases and proteases, respectively.

In another embodiment, the disclosure relates to methods of treatment comprising administering to a subject with a neurological or neuropsychiatric condition susceptible to treatment with digestive enzymes, at least two doses of a composition comprising a therapeutically effective amount of the coated digestive enzyme preparations. In certain embodiments, about 80% of the enzyme is released by about 30 minutes in a dissolution test performed at pH 6.0. In other embodiments, about 80% of the enzyme is released by about 30 minutes after the coated digestive enzyme preparations reach the small intestine.

The disclosure further relates in another aspect to the delivery of digestive enzymes with improved safety of administration. The lipid coat adds weight to the enzyme preparation, which reduces the potential for aerosolization. Previous uncoated enzymes have been shown to become aerosolized, and can therefore be inhaled and contact the nasal cavity or the lungs, causing injury to the mucosa of those taking and those administering the enzyme preparation.

The disclosure further relates to the improvement of administering a sachet preparation for delivery to children. The disclosure specifically relates to the administration of a coated or uncoated digestive enzyme preparation, housed in a sachet which allows for particular types of administration including but not limited to administration in food, drink, or direct administration into the oral cavity or directly into the GI system through a NG-tube, G-tube or other GI entrances. The use of a sachet delivery of enzymes has heretofore not been utilized in the enzyme preparations presently marketed. In one embodiment, the sachet represents a single unit dosage or multiple doses for a day. The sachet of a trilaminar pouch allows the enzyme or enzyme/lipid powder to remain stable, and allows for ease of administration.

The disclosure further relates to the administering of the coated or uncoated enzyme preparation in a sachet or pouch preparation for ease of delivery to children and adults. In some embodiments, the disclosure specifically relates to the administration of a coated or uncoated enzyme particle preparation, housed in a sachet or pouch. This facilitates administration, including but not limited to, administration in food or drink, direct administration into the oral cavity, or administration directly into the GI system through an NG-tube, G-tube or other GI entrances or deliveries.

Compositions comprising an effective amount of the compound may be administered via any conventional route including but not limited to oral, parenteral, intramuscular, intravenous, transmucosal, transdermal, suppository or other method. Further the oral administration can be in the form of pellets, capsules, caplets, beadlets, sprinkles, tablets, softgels or other carrier.

The pharmaceutical formulations can also be prepared for parenteral use. Such formulations typically take the form of sterile isotonic solutions of the active ingredient according to standard pharmaceutical practice.

In one embodiment of the present disclosure, the increase of protein digestion of an individual suffering from a neuropsychiatric disorder leads to the improvement of such disorders. In another embodiment, an individual suffering from or diagnosed with a neuropsychiatric disorder benefits from the administration of digestive enzymes since digestive enzymes aid in the protein digestion process. In one embodiment, the neuropsychiatric symptoms of an individual suffering from or diagnosed with the neuropsychiatric disorder is improved or alleviated from the administration of digestive enzymes.

The present invention provides a method for using digestive enzymes and their derivatives to alleviate the symptoms of neuropsychiatric disorders. The method comprises administering to the individual a digestive enzyme either naturally or recombinantly derived, or their derivatives, in an amount effective to reduce the symptoms of the neuropsychiatric disorder.

Provided herein are methods of preventing one or more symptoms associated with a neuropsychiatric disorder by administering a composition described herein. As used herein, “prevention” refers to prophylaxis, prevention of onset of symptoms, prevention of progression of a neuropsychiatric disorder. As used herein, “inhibition”, “prevention”, “treatment” and “treating” refer to, for example, stasis of symptoms, as well as partial or full amelioration of one or more symptoms associated with a neuropsychiatric disorder. The compositions described herein may be used to minimize the developmental disruption associated with schizophrenia. Patients may be assessed with respect to the prodromal (pre-onset) phase of the illness, which can be detected up to 30 months before the onset of symptoms. Administration of the compositions may be used to limit or reduce the number of patients who go on to develop schizophrenia, experience transient or self-limiting psychotic symptoms and the non-specific symptoms of social withdrawal, irritability, dysphoria, and clumsiness during the prodromal phase.

Compositions can be administered to a patient in an amount that is effective for producing some desired therapeutic effect by alleviating one or more symptoms associated with a neuropsychiatric disorder at a reasonable benefit/risk ratio applicable to any medical treatment. A therapeutically effective amount is an amount achieves at least partially a desired therapeutic or prophylactic effect in tissue subject. The amount of digestive enzymes necessary to bring about alleviation one or more symptoms associated with a neuropsychiatric disorder is not fixed per se. The amount of digestive enzymes administered may vary with the type of disorder, extensiveness of the disorder, and size of the patient suffering from the disorder. A response is achieved when the patient experiences partial or total alleviation, or reduction of one or more signs or symptoms of illness. The patient's symptoms can remain static (i.e., not get worse) or can be reduced.

A physician can readily determine and prescribe the effective amount (ED50) of the composition required. For example, the physician could start doses of the compounds employed in the composition at levels lower than that required in order to achieve the desired therapeutic effect and gradually increase the dosage until the desired effect is achieved. Alternatively, a dose can remain constant.

In such methods of treatment, one or more symptoms are ameliorated or reduced following administration of a composition provided herein. In one embodiment, one or more symptoms of such disorders are reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%. In another embodiment, one or more symptoms of such disorders are reduced in severity or duration by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 90-fold, about 95-fold, about 100-fold or more.

The DSM IV Diagnostic Criteria for Schizophrenia may be used to assess whether administration of a composition described herein reduces the severity and/or duration of one or more symptoms of schizophrenia.

In one embodiment, one or more characteristic symptoms are reduced in severity and/or duration following administration of a composition described herein. For example, two (or more) of the following, each present for less than half or less than a quarter of a 1-month period (or less if successfully treated): delusions, hallucinations, disorganized speech (e.g., frequent derailment or incoherence), grossly disorganized or catatonic behavior, negative symptoms, i.e., affective flattening, alogia, or avolition.

In another embodiment, signs of social/occupational dysfunction are improved following administration of a composition described herein. That is, a patient may exhibit an improvement in one or more major areas of functioning such as work, interpersonal relations, or self-care.

In yet another embodiment, the duration of symptoms may be reduced in severity and/or duration following administration of a composition described herein. That is, continuous signs of disturbance that meet Criterion A may persist for less than 6 months, 5 months, 4 months, 3 months, 2 months, 1 month, 3 weeks, 2 weeks or 1 week.

Another aspect provided herein is combination therapy of a patient with a composition described herein along with another therapeutically effective agent or rehabilitation.

In one embodiment, the one or more agents are an antipsychotic medication. Exemplary antipsychotic medications are dopamine antagonists and serotonin antagonists.

Examples of dopamine antagonists include, but are not limited to: acepromazine, amisulpride, amoxapine, azaperone, benperidol, bromopride, butaclamol, clomipramine (mild), chlorpromazine, chlorprothixene, clopenthixol, clozapine, domperidone, droperidol, eticlopride, flupenthixol, fluphenazine, fluspirilene, haloperidol, iodobenzamide, loxapine, mesoridazine, levomepromazine, metoclopramide, nafadotride, nemonapride, olanzapine, penfluridol, perazine, perphenazine, pimozide, prochlorperazine, promazine, quetiapine, raclopride, remoxipride, risperidone, piperone, spiroxatrine, stepholidine, sulpiride, sultopride, tetrahydropalmatine, thiethylperazine, thioridazine, thiothixene, tiapride, trifluoperazine, trifluperidol, triflupromazine, and ziprasidone.

Examples of serotonin antagonists include, but are not limited to: 5-HT3 antagonists, 5-HT2A receptor antagonists (e.g., ketanserin, which 5 HT2A, 5 HT2C and alpha 1 receptors).

In another embodiment, therapeutically effective rehabilitation includes, but is not limited to, psychotherapy, vocational rehabilitation, social rehabilitation, or a combination thereof.

In another embodiment of the disclosure a transgenic mouse as described by Ratty et. al. (U.S. Pat. No. 5,723,719) and incorporated herein by reference, is utilized to examine the efficacy of the digestive enzyme preparation with respect to administration of an effective amount of the enzyme and evaluating the enzyme's effect on the circling phenotype of the transgenic mouse.

The application of these enzymes of the high protease classification as applied to the chakragati mouse represents a novel discovery for the use of enzymes for neuropsychiatric disorders.

In one embodiment, the digestive enzyme has an effect on the circling behavior and thus on neuropsychiatric disorders including such conditions as schizophrenia.

Studies with mouse models can effectively identify molecules or compounds which may effectively change behaviors in humans. The scale-up of such findings in mice is part of the traditional drug development pathway.

The chakragati (ckr) mouse, the result of a transgenic insertional mutation, exhibits an abnormal circling behavior in response to environmental stress cues, such as cage banging. Wild-type littermates and those that were heterozygous for the transgene insertion did not exhibit this circling phenotype. Analysis of the ckr mouse genome indicates genetic rearrangements on mouse chromosome 16. A US patent was granted for the chakragati mouse (U.S. Pat. No. 5,723,719).

Since its discovery, extensive genetic, pharmacological and behavioral research has been carried out on the ckr mouse. The research undertaken points to the utility of the ckr mouse as a screening tool for neuropsychiatric drug discovery. Cerca Insights Sdn Bhd has developed a cascade of assays, the ckr screen, to characterize the properties of novel antipsychotic compounds. The insights generated from the ckr screen may be used to enhance the assessment of efficacy across the positive, negative and cognitive domains of schizophrenia.

The endophenotypes that have been reported in the literature with respect to the ckr mouse are as follows: asymmetric up-regulation of dopaminergic tone, circling, hyperactivity, prepulse inhibition deficit, latent inhibition (LI), social withdrawal, lateral ventricular enlargement, agenesis of the corpus callosum, and reduction of myelinated neurons in striatum.

The human clinical manifestations of the above mouse endophenotypes are as follows: Left hemi-spatial preference may be linked to asymmetric striatal dopaminergic activity common to all psychoses. A subgroup of schizophrenia patients has underlying right striatal hyper-dopaminergia. There is a greater pathological involvement of the dominant hemisphere in schizophrenia and of the non-dominant hemisphere in bipolar disorder. (Lyon et al. 1992; Bracha 1989; Lohr & Caliguiri 1995).

Yet another human clinical manifestation of the above endophenotypes: Left-prone circling behavior (neglect of right-sided turning) was found in unmedicated schizophrenic patients. A tendency was noted for circling to occur more frequently among paranoid than nonparanoid schizophrenics. (Bracha 1987; Marder & Woods 1987).

Yet another human clinical manifestation of the above endophenotypes: hyperactivation (reduced task-related suppression) of default regions and hyperconnectivity of the default network may contribute to disturbances of thought in schizophrenia and risk for the illness. (Whitfield-Gabrelli et al. 2009).

Yet another human clinical manifestation of the above endophenotypes: impairment in prepulse inhibition (PPI) is generally seen as sensorimotor deficits. PPI disruption occurs in the prodromal stage of schizophrenia and in patients with schizotypical personality disorder. (Quednow et al. 2009; Kumari et al. 2008; Kunugi et al. 2007).

In yet another human clinical manifestation of the above endophenotypes: there is the absence of latent inhibition (LI) in the acute phase of schizophrenia. LI is found to be correlated to the duration of the disease. (Rascle et al. 2001; Gray & Snowden 2005; Vaitl D et al. 2002).

In yet another human clinical manifestation of the above endophenotypes: longer duration of social withdrawal is evident in untreated disease. (Schultz et al. 2007; Iyer 2008; Hoffman 2007).

In yet another human clinical manifestation of the above endophenotypes: ventricular enlargement represents a morphometric endophenotype for schizophrenia. There is a significant correlation between the size of the lateral ventricles and underestimation of the metabolic activity of the caudate. (McDonald et al. 2006; Berkataki et al. 2006; Reig et al. 2007).

In yet another human clinical manifestation of the above endophenotypes: reductions in the thickness of the anterior callosum differentiate between high-risk individuals who transition to psychosis and those who do not, which is highly predictive of transition. (Walterfang et al. 2008).

In yet another human clinical manifestation of the above endophenotypes: myelin impairment is a key factor in the pathogenic loop of psychiatric diseases and drug addiction. (Feng 2008).

The chakragati (ckr) mouse has been utilized as a model for schizophrenia. Many “typical” and “atypical” antipsychotics have been tested on this mouse model prior to, and subsequent to approval in humans.

The circling phenotype exhibits a disruption in the endogenous genetic locus affecting motor function, and the circling behavior may represent an aberration associated with nigrostriatal neurons of the brain. This may affect multiple pathways of neurotransmission such as dopamine, adrenaline, noradrenaline, serotonin or GABA.

The chakragati mouse is a unique and validated animal model which tests “antipsychotic” potency of novel compounds. The mice exhibit a persistent overactivity of the dopamine system caused by a selective increase in striatal D₂ receptor subtypes. In general, antipsychotic medications are thought to reduce dopamine overactivity by the following mechanisms: 1) stimulating dopamine autoreceptors on dopamine neurons, thereby reducing functional activity of the dopamine system and 2) blocking post-synaptic dopamine receptors on dopaminoreceptive neurons or other neurotransmitter systems secondary to dopaminoreceptive neurons.

The chakragati (ckr) mouse has been proposed as a model of aspects of schizophrenia. The mice, created serendipitously as a result of transgenic insertional mutation, exhibit spontaneous circling, hyperactivity, hypertone of the dopamine system, reduced social interactions, enlarged lateral ventricles, deficits in pre-pulse inhibition of acoustic startle and deficits in the latent inhibition of conditioned learning, (Dawe et al. 2010). In 2010 Dawe et al. studied the dose dependent effects of antipsychotic drugs (haloperidol, pimozide, risperidone, clozapine, olanzapine, ziprasidone, quetiapine and aripiprazole) on the spontaneous hyperactivity of the mice. All the antipsychotic drugs tested dose-dependently suppressed spontaneous hyperactivity. Aripiprazole, which is known to be a dopamine D₂ receptor partial agonist, exhibited a tri-phasic dose-response, initially suppressing hyperactivity at low doses, having little effect on hyperactivity at intermediate doses, and suppressing activity again at high doses. These data suggest that the spontaneous circling and hyperactivity of the ckr mouse may allow screening of candidate antipsychotic compounds, distinguishing compounds with aripiprazole-like profiles.

Aripiprazole, (also known as ABILIFY®) is an antipsychotic of a novel class acting as a partial and selective dopamine agonist, produced a different pattern of change in locomotor activity across doses. At lower doses (1.67-10 mg/kg) it produced an apparently dose-dependent reduction in motor activity followed by an increase in motor activity (15 mg/kg) and a subsequent suppression of motor activity (30 mg/kg). It a may be that this multiphasic pattern of change in motor activity across doses reflects the dopamine receptor partial agonist activity of aripiprazole. Thus, the nature of the dose-dependent response in ckr mouse would be expected to differentiate a similar dose-dependent pattern of motor disturbance but the low level of basal activity in wild type mice would make this difficult to detect. Even haloperidol, which is associated with far stronger extrapyramidal motor side effects than aripiprazole, did not produce a significant suppression in the locomotor activity of control mice monitored during the dark cycle when they are most active, (Neuroscience, Dawe et al. 2010).

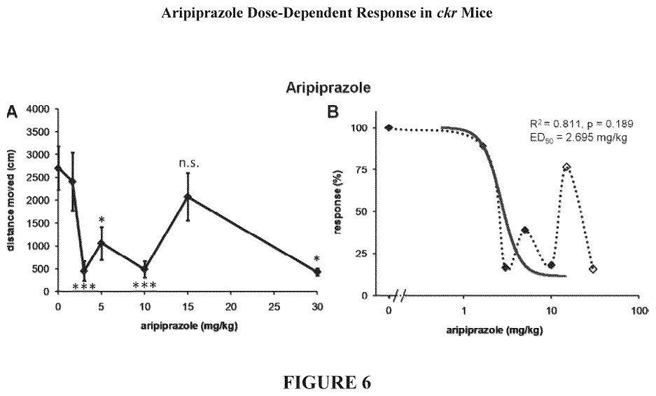

Administration of aripiprazole dose-dependently reduced the locomotor activity of ckr mice (F6, 54_4.626, P_0.001; FIG. 6B). Post-hoc Dunnett's test comparisons with the vehicle control revealed that doses of 3-10 mg/kg significantly reduced locomotor activity. The response appeared to be multiphasic as a higher dose of 15 mg/kg did not significantly suppress locomotor activity while a higher still dose of 30 mg/kg again significantly suppressed locomotor activity. Observationally, the dose of 30 mg/kg appeared to be associated with marked overall suppression of motor function suggestive of severe sedation. Although it was not possible to fit the dose response curve function to the complete data set; it was possible to fit a subset of the data describing the initial suppression of activity at doses from 1.67 to 10 mg/kg with a curve predicting an ED50 of 2.695 mg/kg (FIG. 6B). However, the responses predicted by the function fitted failed to correlate with the actual values observed (R2_0.811, P_0.189), (Dawe et al. 2010).

Thus ckr mice homozygous for the transgene insertion show a constellation of anatomical, biochemical and behavioral deficits which resemble those often reported in schizophrenic patients, as shown in the following Table. (Torres et al. 2008).

| Condition | Schizophrenia | Ckr Mice |
|---|---|---|
| Aberrant Behaviors | | |
| Circling Behavior & Hyperactivity | Yes | Yes |
| Sensorimotor Gating Deficits | Yes | Yes |
| Brain Pathologies | | |
| Ventricular Enlargement | Yes | Yes |
| Myelination Abnormalities | Yes | Yes |
| Metabolic Deficits | Yes | Yes |
| Response to Antipsychotics | | |
| Clozapine and Olanzapine | Yes | Yes |
| Neurochemical Correlates | | |
| Alterations in Dopamine Systems | Yes | Yes |
| Deficit is in Choline and N- | Yes | Yes |
| acetylaspartate | | |
| Typical Age of Onset of Pathology | | |
| Early Adulthood | Yes | Yes** |
| Sex-Dependent Prevalence of Disease | | |

*Human and mice share important genomic, anatomical and physiological similarities. These similarities, particularly in the genes involved in brain development, might provide insight into disease pathogenesis. { . . . }
**(Postnatal Day 10 for onset of Aberrant Behaviors)
(Torres 2008)

The validity of the ckr mouse mutant for understanding the pathogenesis of schizophrenia is further supported in its ability to respond to antipsychotic drug treatment. The most salient endophenotype in the chakragati (ckr) mouse is its circling behavior. Under conditions of subjective stress, the mutant mouse shows consistent circling behavior with individual turns from 10 to 80 full body turns per minute. This behavior syndrome is also characterized by lateral circling behavior (i.e. a left-preference population bias), postural asymmetry and hyperactivity to sensory stimuli. In this context drugs that block the N-Methyl-D-Aspartate (NMDA) subtype of the glutamate receptor such as phencyclidine (PCP, also known as angel dust) and ketamine (a dissociate anesthetic), usually elicit a psychotic-like-state that resembles schizophrenia in preclinical models of the disease. This psychotic-like state includes aberrant behavior syndromes (i.e., positive symptoms) similar to those listed for the ckr mouse. It should be noted that atypical neuroleptics such as clozapine and olanzapine (antipsychotic agents that selectively alleviate symptoms of schizophrenia) also alleviate the lateralized circling behavior and aberrant postural asymmetry exhibited by the ckr mouse. (Torres et al. 2008).

### EXAMPLES

### Example 1. Pancreatin

Pancreatin is a substance comprising enzymes, principally amylase, lipase, and protease, obtained from the pancreas of the hog, Sus scrofa Linné var. domesticus Gray (Fam. Suidae) or of the ox, Bos taurus Linné (Fam. Bovidae). Pancreatin contains, in each mg, not less than 25 U.S.P. units of amylase activity, not less than 2 U.S.P. units of lipase activity, and not less than 25 U.S.P. units of protease activity. Pancreatin of a higher digestive power may be labeled as a whole-number multiple of the three minimum activities or may be diluted by admixture with lactose, or with sucrose containing not more than 3.25 percent of starch, or with pancreatin of lower digestive power.

### Example 2. Pancrelipase

Pancrelipase is a substance containing enzymes, principally lipase, with amylase and protease, obtained from the pancreas of the hog, Sus scrofa Linné var. domesticus Gray (Fam. Suidae). It contains, in each mg, not less than 24 U.S.P. Units of lipase activity, and not less than 100 U.S.P. Units of amylase activity, and not less than 100 U.S.P. Units of protease activity.

The drug substance, pancreatic enzyme concentrate (porcine origin) is purchased from an appropriate supplier. The properties of an exemplary pancreatic enzyme concentrate (pancreatin) suitable for use in the products of this invention are described in the table below.

| Parameter | U.S.P. Specification |
|---|---|
| Protease (U.S.P.) | NLT 25 U.S.P. units/dose |
| Lipase (U.S.P.) | NLT 2 U.S.P. units/dose |
| Amylase (U.S.P.) | NLT 25 U.S.P. units/dose |
| Fat (U.S.P.) | NMT 6.0%* |
| Loss on Drying (U.S.P.) | NMT 5.0% |
| Escherichia coli (U.S.P.) | Neg/10 g |
| Salmonella species (U.S.P.) | Neg/10 g |

*If less than 75 U/mg protease, 6 U/mg lipase or 75 U/mg amylase, then specification is not more than (NMT) 3.0%

### Example 3. Mouse Model Study I

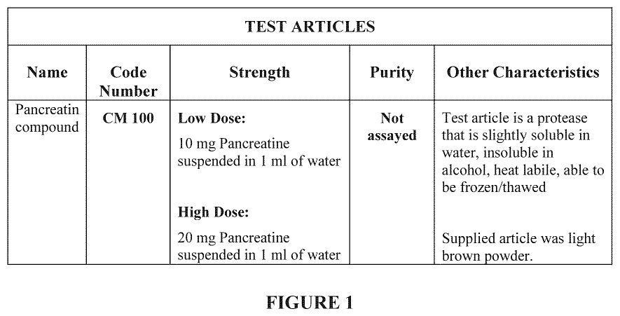

Attenuation of hyperactivity in the ckr mouse has been shown to be predictive of antipsychotic efficacy. By way of testing digestive enzymes to determine their effect on the circling behavior and hyperactivity of the ckr transgenic mouse, two strengths of the enzyme CM100 were tested on the mouse. (FIG. 1). CM100 was administered twice daily for 14 days.

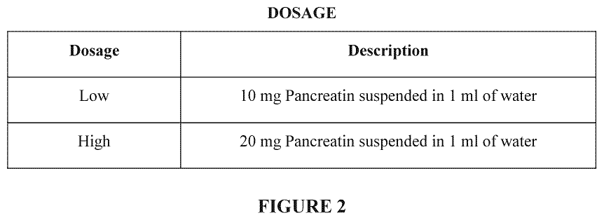

Dosing of the ckr mouse was separated into two strengths: a 10 mg pancreatin suspended in 1 mL of water and a second dosage of 20 mg of pancreatin suspended in 1 mL of water. (FIG. 2). The vehicle used was autoclaved reverse osmosis water. The dosage regimen consisted of twice daily dosing for 14 days at 0800 hours and 2000 hours. Oral gavage was the route of administration. A 20 G autoclavable gavage needle was utilized, and the administration was done at 0.1 ml/10 g BW. The methodology as described may be altered accordingly as one skilled in the art of administration of enzymes or administration via gavage methodology to mice. One ordinarily skilled in the art would be able to administer the enzyme.

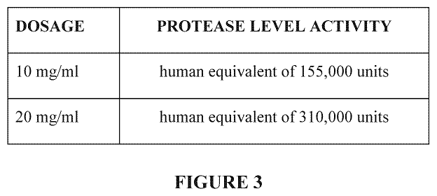

The instant disclosure is comprised of an enzyme preparation comprised of amylases, proteases and lipases. Doses used had a human equivalent strength of between 155,000 and 310,000 units of protease activity, the main component of the enzyme preparation. (FIG. 3).

Dosing of the ckr mouse commenced day 1 and continued to day 14. Dosing was conducted twice daily for 14 days, once in the morning between 7 am and 8 am and once in the evening between 7 pm and 8 pm (12 hours apart). The open field assays were conducted on 6 days: day 1, day 3, day 7, day 14, day 15 and day 18. Distance moved was recorded in 10 min-bins during the 20 minutes monitoring period. Results were compared to the data for the same parameters for 10 minutes determined for untreated ckr mice in prior experiments (n=54).

For the 20 mg/ml (high dose), one-way ANOVA showed significant changes in distance moved, (F6, 87=3.1713, p=0.0076). Paired t-test showed significant differences in velocity between untreated/D1 (t=1.98969, p=0.0011)*, untreated/D3 (t=1.98969, p=0.0454)**, untreated/D7, (t=1.98969, p=0.0157)** and untreated/D18, (t=1.98969, p=0.0203)**. Tukey-Kramer test showed significance between untreated/D1 with p<0.05.

2-way ANOVA showed significant differences in distance moved between low and high dose subjects across time (F1, 14=5.3582, p=0.0363). Within the subjects of high and low dose, there are significant differences across time (F5, 10=4.4951, p=0.0209).

For 20 mg/ml (high dose), one-way ANOVA showed significant changes in velocity (F6, 87=3.1984, p=0.0072). Paired t-test showed significant differences in velocity between untreated/D1 (t=1.98969, p=0.0009)*, untreated/D3 (t=1.98969, p=0.0399)**, untreated/D7, (t=1.98969, p=0.0211)** and untreated/D18, (t=1.98969, p=0.0201)**.

### Example 4. Mouse Model Study II

Dosing of the ckr mouse was separated into two strengths: a 10 mg pancreatin suspended in 1 mL of water and a second dosage of 20 mg of pancreatin suspended in 1 mL of water. (FIG. 2). The vehicle used was autoclaved reverse osmosis water. The dosage regimen consisted of twice daily dosing for 14 days at 0800 hours and 2000 hours. Oral gavage was the route of administration. A 20 G autoclavable gavage needle was utilized, and the administration was done at 0.1 ml/10 g BW. The methodology as described may be altered accordingly as one skilled in the art of administration of enzymes or administration via gavage methodology to mice. One ordinarily skilled in the art would be able to administer the enzyme.

The instant disclosure is comprised of an enzyme preparation comprised of amylases, proteases and lipases. Doses used had a human equivalent strength of between 155,000 and 310,000 units of protease activity, the main component of the enzyme preparation. (FIG. 3).

Dosing commenced day 1 and continued to day 14. Dosing was conducted twice daily for 14 days, once in the morning between 7 am and 8 am and once in the evening between 7 pm and 8 pm (12 hours apart). The open field assays were conducted on 6 days: day 1, day 3, day 7, day 14, day 15 and day 18. Velocity was recorded in 10 min-bins during the 20 minute monitoring period. These were compared to the data for the same parameters for 10 minutes determined for untreated ckr mice in prior experiments (n=54).

For 20 mg/ml (high dose), one-way ANOVA showed significant changes in velocity (F6, 87=3.1984, p=0.0072). Paired t-test showed significant differences in velocity between untreated/D1 (t=1.98969, p=0.0009)*, untreated/D3 (t=1.98969, p=0.0399)**, untreated/D7, (t=1.98969, p=0.0211)** and untreated/D18, (t=1.98969, p=0.0201).

Tukey-Kramer test showed significance between untreated/D1 with p<0.05.

2-way ANOVA showed significant differences in velocity between low and high dose subjects across time (F1, 14=5.0932, p=0.0405). Within the subjects of high and low dose, there are significant differences across time (F5, 10=4.6769, p=0.0184).

### Example 5: Mouse Model Study III

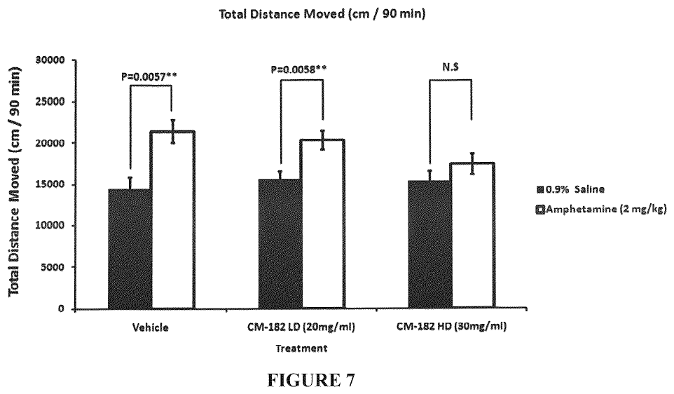

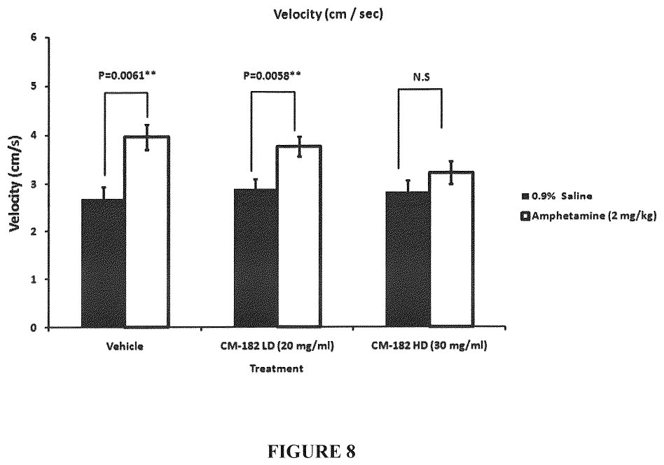

Amphetamine-induced hyperactivity as a model of psychosis in schizophrenia is well established in the industry. FIGS. 7 and 8 are two graphs that depict the results of administration of CM-182 at 30 mg/ml (i.e., approximately 465,000 units of protease) to mice injected with amphetamine to induce hyperactivity and to mice injected with saline as a control.

The dose significantly attenuated the amphetamine-induced hyperactivity, noted as an insignificant difference between the two groups of mice in total distance moved in FIG. 7 and in velocity in FIG. 8. It was also observed that CM-182 did not affect baseline activity in these mice, suggesting that the attenuation of hyperactivity occurred in a manner unrelated to sedation. Essentially, the administered compound is non-sedating. What the present inventors have identified is that significance exists between the two of the groups in both graphs (but not in the 30 mg/ml group), suggesting that the amphetamine did indeed induce hyperactivity to begin with.

While preferred embodiments have been shown and described herein, such embodiments are provided by way of example only. Numerous variations, changes and substitutions may occur without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-99. (canceled)

100. A method for treating schizophrenia in a subject in need thereof, comprising orally administering to the subject a dose of a therapeutically effective amount of a pharmaceutical composition comprising digestive enzymes; wherein the digestive enzymes comprise a protease, an amylase, and a lipase, and wherein the dose of the pharmaceutical composition comprises from about 5,000 to about 1,000,000 U.S.P. units of protease activity, whereby schizophrenia is treated.

101. The method of claim 100, wherein the digestive enzymes comprise a protease, an amylase, and a lipase.

102. The method of claim 101, wherein the protease and the lipase are present in the pharmaceutical composition in ratio of total protease to total lipase in U.S.P. units, and wherein the ratio is from about 5.371 to about 20:1.

103. The method of claim 101, wherein the protease is present in the pharmaceutical composition in an amount of about 140,000 U.S.P. units/dose, the amylase is present in the pharmaceutical composition in an amount of about 144,000 U.S.P. units/dose, and the lipase is present in the pharmaceutical composition in an amount of about 23,000 U.S.P. units/dose.

104. The method of claim 100, wherein the pharmaceutical composition is a dosage formulation selected from the group consisting of a capsule, a tablet, a sprinkle, a powder, a pellet, and a caplet.

105. The method of claim 100, wherein the method further comprises administering an antipsychotic medication to the subject.

106. The method of claim 105, wherein the antipsychotic medication is a dopamine antagonist or a serotonin antagonist.

107. The method of claim 100, wherein the method further comprises administering to the subject psychotherapy, vocational rehabilitation, social rehabilitation, or a combination thereof.

108. A method for treating schizophrenia in a subject in need thereof, comprising orally administering to the subject a therapeutically effective amount of a pharmaceutical composition, wherein the composition comprises digestive enzymes coated with a lipid coating or an enteric coating; and wherein the digestive enzymes comprise a protease, an amylase, and a lipase, whereby schizophrenia is treated.

109. The method of claim 108, wherein the digestive enzymes comprise a protease, an amylase, and a lipase.

110. The method of claim 108, wherein the digestive enzymes are coated with the enteric coating.

111. The method of claim 108, wherein the digestive enzymes are coated with the lipid coating.

112. The method of claim 111, wherein the lipid coating comprises an animal lipid or a vegetable lipid.

113. The method of claim 111, wherein the lipid coating comprises monoglycerides, diglycerides, triglycerides, fatty acids, esters of fatty acids, phospholipids, salts thereof, or a combination thereof.

114. The method of claim 111, wherein the digestive enzymes are in the form of coated particles.

115. The method of claim 114, wherein the digestive enzymes are present in the coated particles in an amount of from about 75% to about 85% by weight.

116. The method of claim 114, wherein the digestive enzymes are present in the coated particles in an amount of from about 77.5% to about 82.5% by weight.

117. The method of claim 114, wherein the digestive enzymes are present in the coated particles in an amount of about 70%, about 75%, about 77.5%, about, 80%, about 82.5%, about 85%, or about 90% by weight.

118. The method of claim 111, wherein the lipid coating comprises a hydrogenated soy oil.

119. A composition comprising digestive enzymes for use in the treatment of a neuropsychiatric disorder.
